(19) United States
(12) Patent Application Publication
(10) Pub. No.: US 2021/0095259 A1
(43) Pub. Date: Apr. 1, 2021

(54) COMPOSITION AND METHOD FOR INDUCTION OF PLURIPOTENT STEM CELL

(71) Applicant: GMU Medical Drug Development Co., Ltd., Guangzhou (CN)

(72) Inventors: Xiyong YU, Guangzhou (CN); Jialiang LIANG, Guangzhou (CN); Yigang WANG, Guangzhou (CN)

(21) Appl. No.: 17/118,555

(22) Filed: Dec. 10, 2020

Related U.S. Application Data
(63) Continuation-in-part of application No. PCT/CN2019/073269, filed on Jan. 25, 2019.

(30) Foreign Application Priority Data
Jul. 6, 2018 (CN) 201810739342.3

(51) Int. Cl. C12N 5/074

(52) U.S. Cl. CPC C12N 5/0696; C12N 2510/00; C12N 2501/727; C12N 2501/15; C12N 2501/65; C12N 2501/065

(57) ABSTRACT

A composition for induction of a pluripotent stem cell includes a microRNA and a combination of small-molecule compounds. The combination of small-molecule compounds includes a histone deacetylase inhibitor, a mitogen-activated extracellular signal-regulated kinase inhibitor, a glycogen synthase kinase-3 inhibitor, a transforming growth factor beta receptor 1 (TGF-βR1) inhibitor, an inhibitor of histone demethylase.

### CROSS-REFERENCE TO RELATED APPLICATIONS

This application is a continuation-in-part of International Patent Application No. PCT/CN2019/073269 with an international filing date of Jan. 25, 2019, designating the United States, now pending, and further claims foreign priority benefits to Chinese Patent Application No. 201810739342.3 filed on Jul. 6, 2018. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND

The disclosure relates to the field of cell culture, and more particularly, to a composition and method for induction of a pluripotent stem cell.

Induced pluripotent stem cells (also known as iPS cells or iPSCs) are a type of pluripotent stem cell that can be generated directly from a somatic cell. microRNA (miRNA) is a small molecule encoded by the genomes of higher eukaryotes. miRNAs can be coupled with target mRNAs through base pairing to activate RNA-induced silencing complex (RISC) that degrades mRNAs or prevents mRNAs from being translated.

miRNAs have been found to be highly conserved among species during evolution. miRNAs are expressed only in specific tissues or developmental stages of plants, animals, and fungi, indicating that miRNAs have important biological functions and determine the functional specificity of tissues and cells during cell growth and development. In addition to pluripotent transcription factors, endogenous specific microRNAs (miRs) are highly expressed in embryonic stem cells, and therefore are known as ES-cell-specific miRNAs. miRs have been found to control the expression of pluripotency-related genes, hence miRs is of importance in self-renewal, differentiation, and dedifferentiation of cells. miRs are small non-coding RNA molecules that generally function as an endogenous inhibitor to control the post-transcriptional modification by degrading mRNA or preventing mRNA from being translated. A given miR may inhibit hundreds of different mRNA targets, and a given target may be regulated by multiple miRNAs, resulting in distinct changes in gene expression profiles and cell phenotypes.

Although certain small-molecule compounds for example, valproic acid and tranylcypromine, have been found to have the ability to induce somatic cells into a pluripotent state, the process is inefficient.

### SUMMARY

In the disclosure, the miR-290 family members are used in combination with valproic acid (a histone deacetylase inhibitor), RepSox (E-616452, a transforming growth factor beta receptor 1 inhibitor and a histone demethylase inhibitor), CHIR-99021 (a glycogen synthase kinase-3 inhibitor), PD0325901 (a mitogen-activated extracellular signal-regulated kinase inhibitor), and tranylcypromine (an inhibitor of LSD1 histone demethylase and monoamine oxidases (MAO)).

The disclosure provides a composition for induction of iPS cells.

The composition comprises a microRNA and a combination of small-molecule compounds. The combination of small-molecule compounds comprises a histone deacetylase inhibitor, a mitogen-activated extracellular signal-regulated kinase inhibitor, a glycogen synthase kinase-3 inhibitor, a transforming growth factor beta receptor 1 (TGF-βR1) inhibitor, an inhibitor of histone demethylase.

The disclosure provides a method for induction of a pluripotent stem cell using the composition, the method comprising transfecting the microRNA of the composition into a somatic cell during reprogramming of the somatic cell, and adding synchronously or later the small-molecule compounds of the composition.

Specifically, the composition comprising the pluripotent microRNA (micro-ribonucleic acid or small-molecule ribonucleic acid) and the combination of small-molecule compounds induces the somatic cells into the iPS cells. The induction process involves no exogenous transcription factor linked to virus, thus preventing the risk of mutations caused by gene integration and improving the safety of preparation of iPS cells.

### BRIEF DESCRIPTION OF THE DRAWINGS

FIG. 1 is a schematic diagram of introducing miR-290 family members into SkMC to yield iPS cells. SkMC: skeletal muscle cell; MEF: mouse embryonic fibroblast; ALP: alkaline phosphatase; GFP: green fluorescent protein; OKSM: a combination of Oct4, Klf4, Sox2 and c-Myc; KSM: a combination of Klf4, Sox2 and c-Myc.

FIG. 2A shows Oct4-GFP expression and AP staining of iPS cell colonies; FIGS. 2B and 2C shows generation efficiency of iPS cell analyzed by ALP staining, where the iPS cells are transfected with OSKM in combination with the mimics (B) or inhibitors (C) of miR-290 family members.

FIGS. 3A and 3B show ALP staining depicting the number of iPS cell colonies generated by miR-291a in combined with different reprogramming factors; FIG. 3C shows ALP-positive iPS cell colonies.

FIG. 4A shows immunofluorescence staining of iPS cells differentiating into three germ layer cells in vitro; FIG. 4B shows HE staining depicting the ability of iPS cells to differentiate to form teratomas in vivo.

FIG. 5 shows immunofluorescence staining of iPS cells in vitro differentiating into cardiomyocytes positive for cTnT or α-actinin. The iPS cells generated by MiR-291a+SKM differentiate into cardiomyocytes.

FIG. 6 shows cardiomyocytes differentiated from iPS cells induced by miR-291a+SKM improve myocardial function in mice with myocardial infarction.

FIG. 7 is a schematic diagram of transfecting miR-291a+VC6TP into somatic cells to generate iPS cells. VC6TP refers to a combination of small-molecule compounds comprising VPA (V), CHIR99021 (C), E-616452 (6), Tranylcypromine (T), and PD0325901 (P).

FIG. 8A shows positive GFP expression and AP staining of iPS cell colonies at 10 days after induction of miR-291a and VC6TP. The iPS cells selected for clonal culture continuously express the GFP or ALP marker; FIG. 8B shows the number of ALP-positive iPS cells generated by different combinations of small-molecule compounds.

FIG. 9 shows immunofluorescence staining of iPS cells generated by MiR-291a+VC6TP that expresses pluripotent markers.

FIG. 10A shows immunofluorescence staining of iPS cells generated by MiR-291a+VC6TP that are differentiable into three germ layer cells in vitro; FIG. 10B shows HE staining depicting the ability of iPS cells generated by MiR-291a+VC6TP that are differentiable to form teratomas in vivo.

### DETAILED DESCRIPTION

To further illustrate the disclosure, embodiments detailing a composition for induction of iPS cells are described below. It should be noted that the following embodiments are intended to describe and not to limit the disclosure.

The microRNA of the disclosure is a micro-ribonucleic acid or small-molecule ribonucleic acid.

The microRNA of the disclosure comprises one of the miR-290 family members, or a microRNA sequence having at least 90% identity with the sequence of one of the miR-290 family members.

The microRNA is mmu-mir-290a, mmu-mir-291a, mmu-mir-291b, mmu-mir-292a, mmu-mir-293, mmu-mir-294, mmu-mir-295, bta-mir-371, cfa-mir-371, eca-mir-371, eca-mir-372, ggo-mir-371b, hsa-mir-371 a, hsa-mir-371b, hsa-mir-372, mml-mir-371, mml-mir-371-2, mml-mir-372, ocu-mir-371, ocu-mir-373, ppy-mir-371, ppy-mir-372, ptr-mir-371, ptr-mir-372, rno-mir-290, rno-mir-291 a, rno-mir-291b, rno-mir-292, rno-mir-293, rno-mir-294, rno-mir-295-1, rno-mir-295- 2, ssc-mir-371, or a combination thereof.

The histone deacetylase inhibitor is a compound that reduces or inhibits the activity of histone demethylases (HDACs). The histone deacetylase inhibitor is valproic acid, trichostatin A (TSA), M344 (a HDAC inhibitor), sodium phenylbutyrate, entinostat (MS-275), belinostat (PXD101), abexinostat (PCI-24781), dacinostat (LAQ824), quisinostat (JNJ-26481585) 2HC1, mocetinostat (MGCD0103), droxinostat, MC1568, pracinostat (SB939), divalproex sodium, PCI-34051, givinostat (ITF2357), AR-42, tubastatin A HC1, resminostat, tacedinaline (CI994), RGFP966, HPOB, RG2833 (RGFP109), TMP269, nexturastat A, 4SC-202, LMK-235, or a combination thereof.

In certain embodiments, the mitogen-activated extracellular signal-regulated kinase inhibitor is a compound that reduces or inhibit the activity of mitogen-activated extracellular signal-regulated kinase (MEK). The mitogen-activated extracellular signal-regulated kinase inhibitor is PD0325901, selumetinib (AZD6244), PD184352 (CI-1040), SL-327, refametinib (RDEA119, Bay 86-9766), PD98059, pimasertib (AS-703026), BIX 02188, BIX 02189, PD318088, AZD8330, myricetin, TAK-733, trametinib (GSK1120212), binimetinib (MEK162, ARRY-162, ARRY-438162), GDC-0623, Cobimetinib (GDC-0973, RG7420), APS-2-79 HC1, derivatives thereof, or a combination thereof.

The glycogen synthase kinase-3 inhibitor is a compound that reduces or inhibits the activity of glycogen synthase kinase-3 (GSK-3). The glycogen synthase kinase-3 inhibitor is CHIR-99021 (CT99021), SB216763, TWS119, indirubin, SB415286, CHIR-98014, Tideglusib, TDZD-8, LY2090314, AZD1080, 1-Azakenpaullone, BIO, AZD2858, AR-A014418, IM-12, bikinin, BIO-Acetoxim, or a combination thereof.

The transforming growth factor beta receptor 1 (TGF-βR1) inhibitor is a compound that reduces or inhibits the activity of transforming growth factor beta receptor 1 (TGF-βR1) or blocks the TGF-β signal transduction pathway. The transforming growth factor beta receptor 1 (TGF-βR1) inhibitor is RepSox (E-616452), SB-431542, SB-525334, theophylline, SB-505124, galunisertib (LY2157299), GW-788388, pirfenidone, DMH1, LDN-212854, K02288, vactosertib (TEW-7197), SD-208, LDN-214117, SIS3 HC1, LDN-193189 2HC1, or a combination thereof.

The inhibitor of histone demethylase is a compound that reduces or inhibits the activity of histone demethylases. The inhibitor of histone demethylase is tranylcypromine, GSK J4HC1, IOX1, OG-L002, JIB-04, ML324, GSK-LSD1 2HC1, GSK J1, SP2509, ORY-1001(RG-6016) 2HC1, GSK2879552 2HC1, CPI-455HC1, or a combination thereof.

A combination of small-molecule compounds comprises valproic acid, PD0325901, CHIR-99021, E-616452, and tranylcypromine.

Preferably, the amount of each of the small-molecule compounds is as follows: 10±1 nM valproic acid, 1±0.1 nM CHIR-99021, 1±0.1 nM E-616452, 5±0.5 nM tranylcypromine, and 1±0.1 nM PD0325901.

In certain embodiments, the somatic cells comprise human dermal fibroblasts, blood cells, skeletal muscle cells, bone marrow mononuclear cells, and mesenchymal stem cells; preferably, the somatic cells are human skeletal muscle cells.

In certain embodiments, a method for induction of a pluripotent stem cell using the composition, the method comprising transfecting the microRNA of the composition into a somatic cell during reprogramming of the somatic cell, and adding synchronously or later the small-molecule compounds of the composition.

### EXAMPLE 1

microRNA was transfected into somatic cells to generate iPS cells.

1. Isolation and culture of somatic cells:

Skeletal muscle cells (SkMCs) were isolated from limb muscles of 8-10-week-old Oct-4-GFP transgenic mice (acquired from Jackson Lab). Mouse muscle tissues (up to 100 mg) were washed, cut into small pieces, dissociated with DMEM containing 0.1% collagenase II in a 37° C. water bath for 2 hours, digested with 0.15% trypsin for 45 minutes, followed by the addition of 10% fetal bovine serum (FBS) to inactivate the collagenase/trypsin. The resulting cell suspension was then filtered using a 70 μm cell strainer, centrifuged at 2000 rpm for 5 minutes, washed with phosphate buffered saline (PBS), and resuspended in 10 mL of SkMC growth medium (high-glucose DMEM contains 0.1 mM non-essential amino acids, 10 ng/mL bone morphogenic protein 4 (BMP-4), 10% FBS and 2.5% penicillin/streptomycin). Primary cells within five generations were used in all experiments.

2. Cell reprogramming: following the method of Yamanaka et al. (Cell, 2007, 131(5): 861-872.), HEK293T cells were transfected with a retroviral vector (41 μg) comprising a packaging plasmid pCL-Eco (41 μg) and OKSM reprogramming factors. The retroviral vector comprises pMXs-K1f4, pMXs-Sox2, pMXs-Oct4, or pMXs-c-Myc (purchased from Addgene, Inc). Retrovirus was purchased from Addgene, Inc. The cell culture supernatant of the HEK293T cells were harvested at two days after transfection, filtered by a 0.45 μm filter and stored at −80° C., thereby obtaining a retroviral vector supernatant. Mouse miR-291a and its mimics were purchased from Dharmacon, Inc. SkMCs (at a density of 1×10⁵ cells per well and passage 3-5) were plated into a six-well plate (day 0), and infected with the retrovirus vector (the ratio of retroviral vector supernatant and medium is 1:2). 24 hours later, the medium was replaced with SkMC medium for the transfection of miR-291a and its mimics. Specifically, 100 nM mouse miR-291a and its mimics were respectively mixed with 4 μL of Dharmacon transfection reagent and added to the SkMC medium. After incubation for 2 days, the SkMCs (1×10⁴ ) were transferred to a 6 cm petri dish covered with a layer of mouse embryonic fibroblasts, and incubated in a knockout DMEM medium (Life Technologies) containing 15% FBS, 0.1 mM non-essential amino acids, 0.1 mM GlutaMAX, 0.1 mM b-mercaptoethanol, and LIF (1,000 U/mL). The knockout DMEM medium was changed every two days. The SkMC change was monitored continuously. After incubation for 2-3 weeks, the total number of iPS cell colonies was determined.

3. Identification of cell pluripotency: (1) Alkaline phosphatase (ALP) staining: the iPSC colonies were stained using ALP Live Cell Staining Kit (purchased from ThermoFisher Scientific) according to the manufacturer's instructions, observed and counted under a microscope. (2) Immunofluorescence staining: the iPS cells growing on a glass slide were fixed with 4% paraformaldehyde for 10-20 minutes, permeabilized with a blocking buffer containing 0.1% bovine serum albumin (and 0.1% triton X-100) for 10 to 30 minutes, washed with PBS, incubated with primary antibody (purchased from ABcam, Inc) overnight at 4° C., followed by removal of primary antibody. Then the iPS cells was reacted with a fluorescein-labeled secondary antibody at room temperature for 1 hour. Nuclei were stained with 4′,6-diamidino-2-phenylindole (DAPI) and viewed under the microscope. (3) Teratoma assay: undifferentiated iPS cells were collected, resuspended in Hank's Balanced Salt Solution, loaded into a pre-cooled syringe with a 27-gauge needle, and subcutaneously injected into NU/J mice (purchased from Jackson Lab). The NU/J mice were sacrificed at 4 weeks after injection, followed by tissue preparation for immunofluorescence staining.

4. Cellular differentiation: iPS cell colonies were collected, digested with Dispase at 37° C. for 3-5 minutes, plated into an ultra-low attachment plate, suspended in a differentiation medium, and incubated for 7 days to yield a cell suspension. The differentiation medium contained 0.1 mM non-essential amino acids, 1 mM L-glutamine, 0.1 mM dimercaptoethanol, 20% FBS, and high-glucose DMEM. An embryoid body was formed, transferred onto a petri dish coated with 0.1% gelatin, and incubated for one week.

5. Validation of myocardial infarction model: the left anterior descending coronary artery of 10-12-week-old mice was ligated, thereby building a mouse myocardial infarction model (Ma, R., et al. (2018). Antioxid Redox Signal 28(5): 371-384.). 30 μL of the cell suspension was subcutaneously injected three times into three different areas of the border zone of the myocardial infarction. The changes of myocardial function in the mice was examined by echocardiography (iE33Ultrasound System; Phillips) at four weeks after surgery.

6. Statistical analysis: statistical analysis was performed with SPSS version 13.0 statistic software. T-test was used to determine if the means of two sets of data were significantly different from each other. One-way analysis of variance (ANOVA) was used when the data belong to more than two sets. A p-value less than 0.05 (p<0.05) was statistically significant.

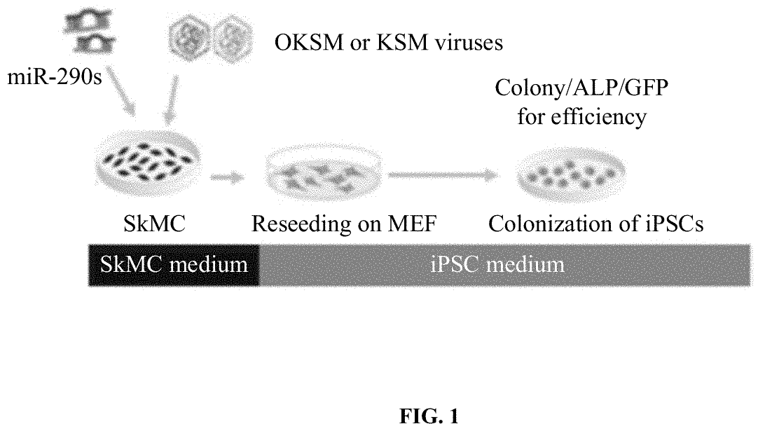

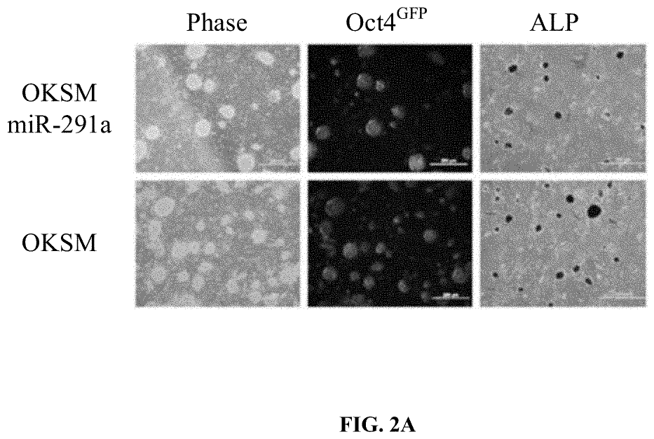

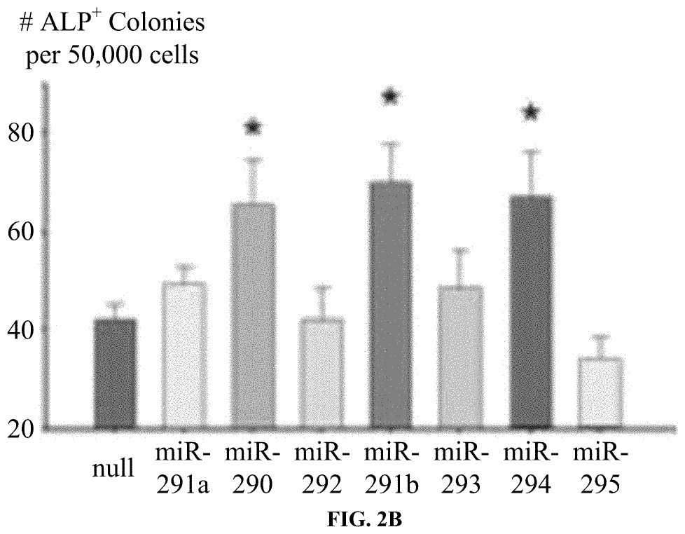

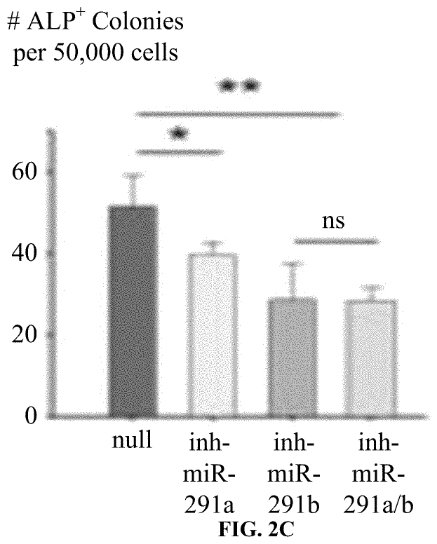

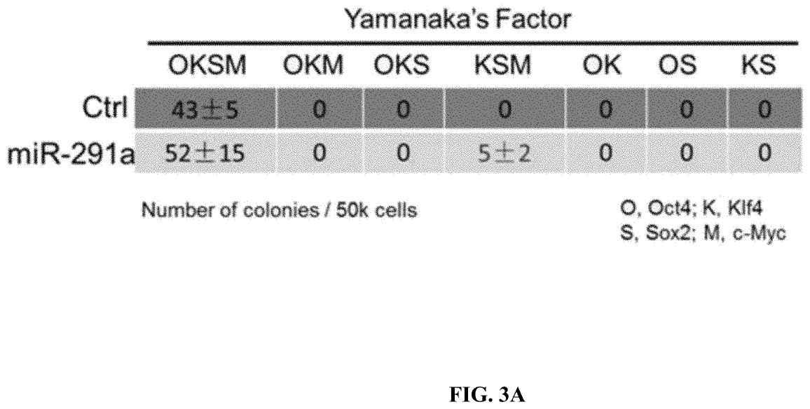

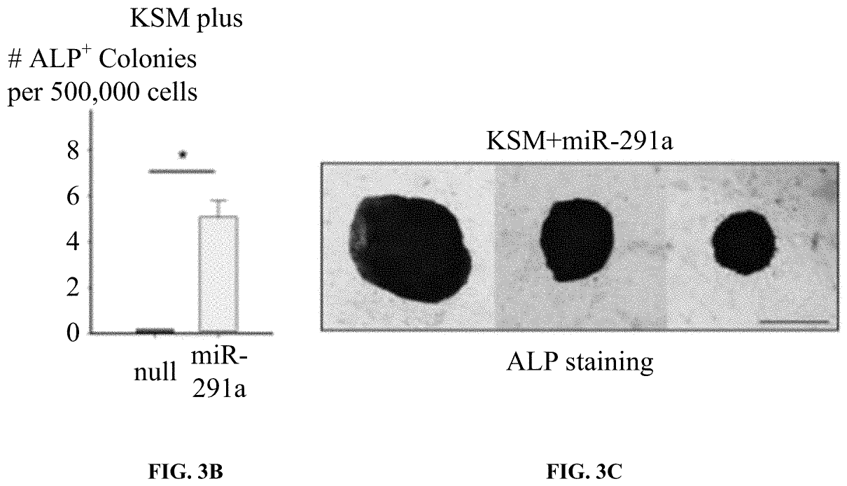

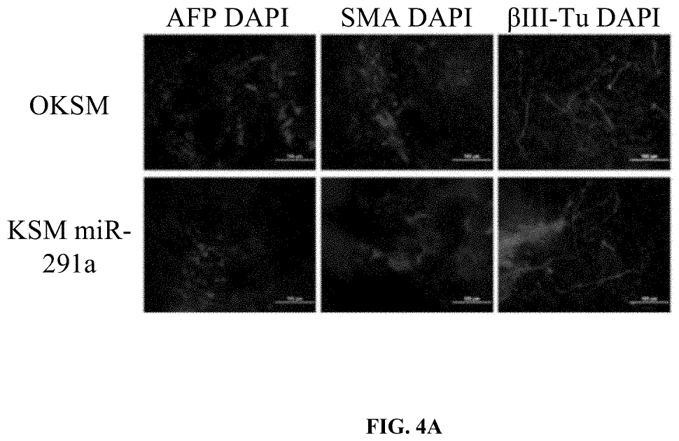

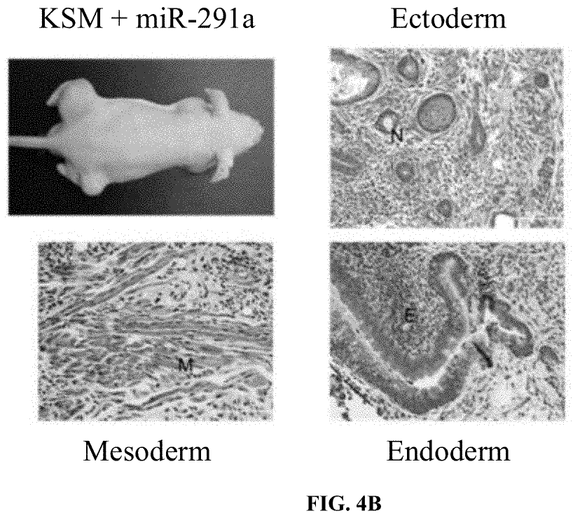

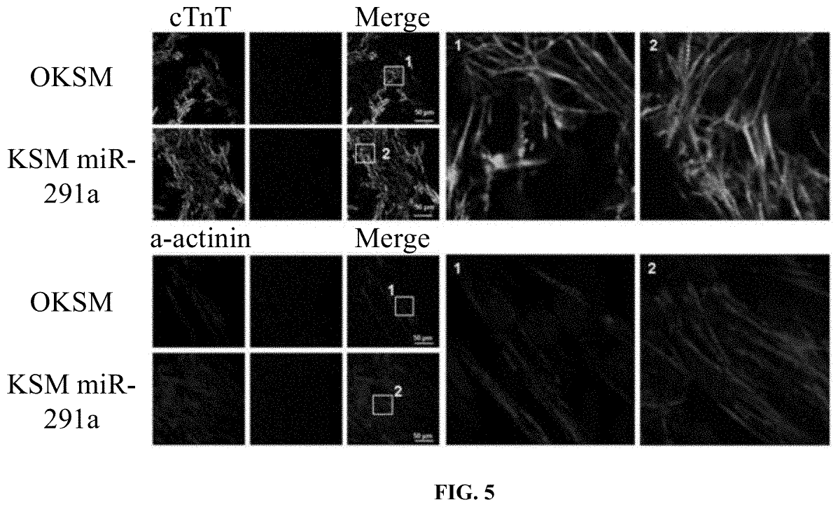

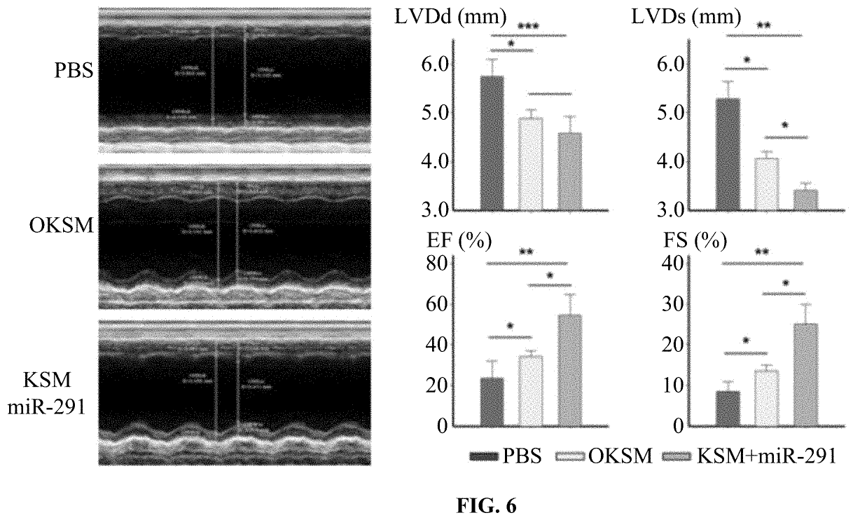

7. Experiment results: FIG. 1 is a schematic diagram of transfecting miR-290 family members into SkMCs to generate iPS cells. iPS colonies were formed and observed at 2 weeks after transfection. Introduction of miR-291a further enhanced the reprogramming efficiency of OKSM, increased the number of Oct4-GFP-positive and ALP-positive colonies (as shown in FIG. 2). Similar effects were observed with the introduction of other miR-290 family members. For example, OKSM achieved increased reprogramming efficiency by combining with miR-291b or miR-294 (FIG. 2B), and reprogrammed at lower efficiency when combining with miR-291a inhibitor or miR-291b inhibitor (FIG. 2C). The results indicated that miR-291a and miR-291b were involved in the reprogramming process of OKSM. OKSM refers to four reprogramming factors including Oct3/4, Sox2, Klf4, and c-Myc. The S134Cs when treated with miR-291a+KSM yielded iPS cells (FIGS. 3A and 3B), while the S134Cs treated with KSM alone yielded none. The results indicated that miR-291a could replace Oct4 or other reprogramming factors in promoting the reprogramming of somatic cells into iPS cells. The morphology of iPS cells induced by miR-291a+KSM is shown in FIG. 3C. FIG. 4A shows immunofluorescence staining of iPS cells differentiating into three germ layer cells in vitro. The iPS cells induced by KSM+miR-291a had similar functions to that induced by OKSM, and possessed the multipotent differentiation, being able to spontaneously differentiate into three germ layer cells, including alpha fetoprotein (AFP) positive endoderm-like cells, alpha-smooth muscle actin (α-SMA) positive mesoderm-like cells, and β-III Tubulin Tubulin, β-III Tu) positive ectoderm-like cells. Four weeks after injection of the iPS cells into immunodeficient mice, mouse tissue was stained with haematoxylin and eosin and demonstrated that the iPS cells had the potential to differentiate into any of the three germ layer cells. Referring to FIG. 4B, the iPS cells induced by miR-291a+KSM induced teratomas formation with differentiation down endoderm intestinal epithelial tissue (E), mesoderm muscle-like tissue (M), and ectoderm nerve rosette-like structure (N). The iPS cells was further explored for cardiovascular regeneration in medical applications. Referring to FIG. 5, the iPS cells generated by miR-291a+KSM could differentiate into cardiomyocyte-like cells and expressed proteins related to the cardiomyocytes in contractile function, such as troponin T (cTnT) and actinin (α-actinin). The cardiomyocytes differentiated from the iPS cells were injected into the border zone of the myocardial infarction. The echocardiography demonstrates that the iPS cells induced by KSM+miR-291a produced better therapeutic effect on myocardial infarction than that of the control group and OKSM. Introduction of KSM+miR-291 a significantly increased cardiac performance parameters such as ejection fraction (EF) and shortening fraction (FS) (FIG. 6).

### EXAMPLE 2

iPS cells were induced by microRNA and small-molecule compounds.

1. Isolation and culture of somatic cells: somatic cells were obtained in the same manner as in Example 1.

2. Cell programming: SkMCs (at a density of 1×10⁵ cells per well, passage 3-5) were plated into a six-well plate (day 0), and transfected with miR-291a 24 hours after plating. Specifically, 100 nM mouse miR-291a was mixed with 4 μL of Dharmacon transfection reagent, added to a SkMC medium, and added to the six-well plate. After incubation for 2 days, the SkMCs (1×10⁴) were transferred to a 6 cm petri dish covered with a layer of mouse embryonic fibroblasts, and incubated in a knockout DMEM medium (Life Technologies) containing 15% FBS, 0.1 mM non-essential amino acids, 0.1 mM GlutaMAX, 0.1 mM b-mercaptoethanol, and LIF (1,000 U/mL). The small-molecule compounds at optimal concentrations were subsequently added to the petri dish (the small-molecule compounds (hereafter, VC6TP) contained 10 nM VPA, 1 nM CHIR-99021, 1 nM E-616452, 5 nM Tranylcypromine, and 1 nM PD0325901, all of which were purchased from Sigma). The knockout DMEM iPSC medium was changed every two days. The SkMC change was monitored continuously. After incubation for 2-3 weeks, the total number of iPSC colonies was determined.

3. Identification of cell pluripotency: cell pluripotency was identified in the same manner as in Example 1.

4. Statistical analysis: statistical analysis was performed with SPSS version 13.0 statistic software. One-way analysis of variance (ANOVA) was used when the data belong to more than two groups. A p-value less than 0.05 (p<0.05) was statistically significant.

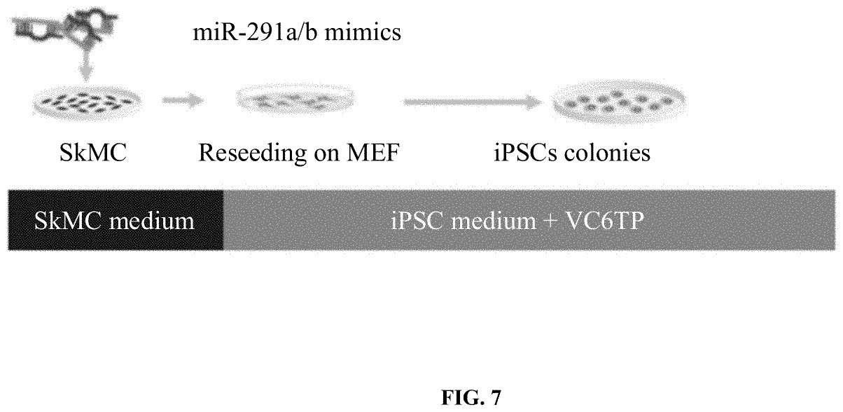

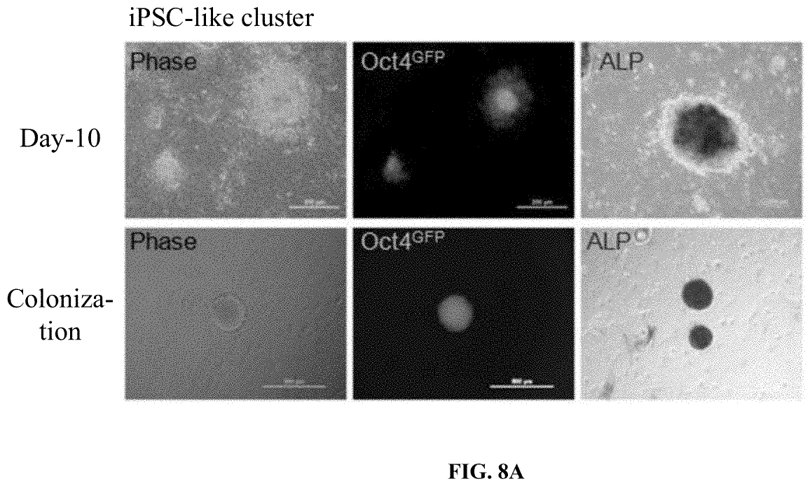

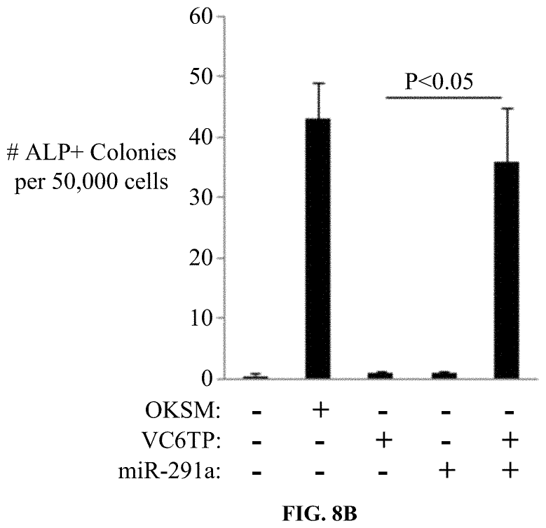

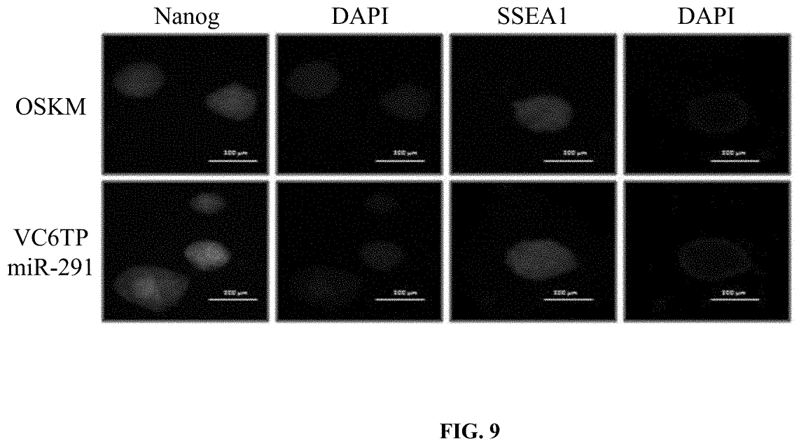

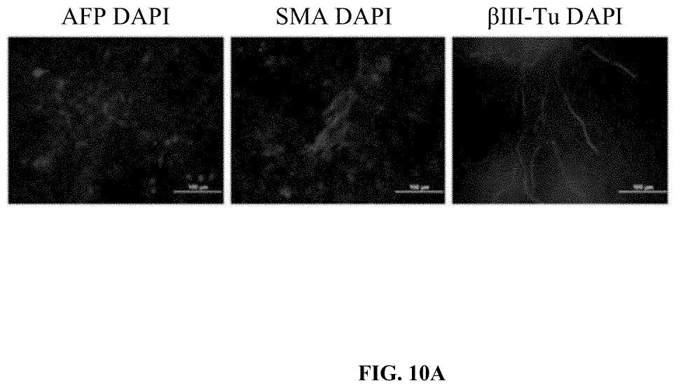

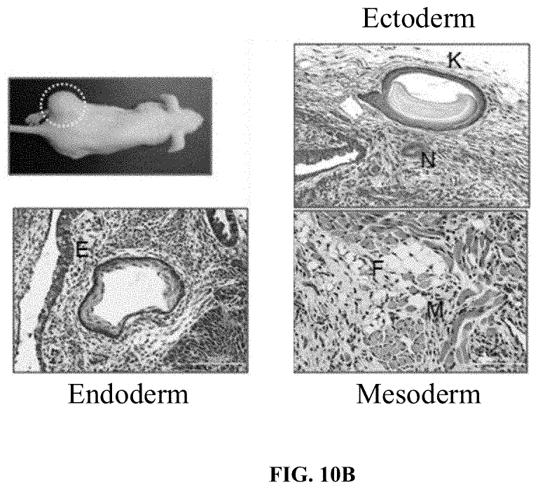

5. Experiment results: FIG. 7 a schematic diagram of reprogramming somatic cells into iPS cells with miR-291a+VC6TP. iPS colonies were formed and observed at 2 weeks. miR-291a+VC6TP induced SkMC to generate an iPS cell colony stained positive for Oct4-GFP and ALP (FIG. 8A), while the SkMCs treated with miR-291a or VC6TP alone yielded none. miR-291a+VC6TP possessed a reprogramming efficiency comparable to that of OKSM (as shown in FIG. 8B). Referring to FIG. 9, immunofluorescence confirmed that other pluripotency markers were simultaneously expressed in the iPS cells produced by miR-291a+KSM, such as Nanog and Sseal. The iPS cells yielded by miR-291a+KSM had similar functions to that induced by OKSM, and possess multipotent differentiation, being able to spontaneously differentiate into three germ layer cells, including alpha fetoprotein (AFP) positive endoderm-like cells, alpha-smooth muscle actin (α-SMA) positive mesoderm-like cells, and β-III Tubulin (β-III Tubulin, β-III Tu) positive ectoderm-like cells. Four weeks after injection of the iPS cells into immunodeficient mice, mouse tissue was stained with haematoxylin and eosin and shows that the iPS cells have the potential to differentiate into any of the three germ layer cells. Referring to FIG. 10B, the iPS cells yielded by miR-291a+KSM induced teratomas formation with differentiation down endoderm intestinal epithelial tissue (E), mesoderm muscle-like tissue (M) or adipose-like tissue (F), ectoderm nerve rosette-like structure (N) and keratinized bead structure (K).

It will be obvious to those skilled in the art that changes and modifications may be made, and therefore, the aim in the appended claims is to cover all such changes and modifications.

What is claimed is:

1. A composition, comprising a microRNA and a combination of small-molecule compounds; wherein the combination of small-molecule compounds comprises a histone deacetylase inhibitor, a mitogen-activated extracellular signal-regulated kinase inhibitor, a glycogen synthase kinase-3 inhibitor, a transforming growth factor beta receptor 1 (TGF-βR1) inhibitor, an inhibitor of histone demethylase.

2. The composition of claim 1, wherein the microRNA comprises one of the miR-290 family members, or a microRNA sequence having at least 90% identity with a sequence of one of the miR-290 family members.

3. The composition of claim 2, wherein the microRNA is mmu-mir-290a, mmu-mir-291 a, mmu-mir-291b, mmu-mir-292a, mmu-mir-293, mmu-mir-294, mmu-mir-295, bta-mir-371, cfa-mir-371, eca-mir-371, eca-mir-372, ggo-mir-371b, hsa-mir-371a, hsa-mir-371b, hsa-mir-372, mml-mir-371, mml-mir-371-2, mml-mir-372, ocu-mir-371, ocu-mir-373, ppy-mir-371, ppy-mir-372, ptr-mir-371, ptr-mir-372, rno-mir-290, rno-mir-291a, rno-mir-291b, rno-mir-292, rno-mir-293, rno-mir-294, rno-mir-295-1, rno-mir-295- 2, ssc-mir-371, or a combination thereof.

4. The composition of claim 1, wherein the histone deacetylase inhibitor is valproic acid, trichostatin A (TSA), M344 (a HDAC inhibitor), sodium phenylbutyrate, entinostat (MS-275), belinostat (PXD101), abexinostat (PCI-24781), dacinostat (LAQ824), quisinostat (JNJ-26481585) 2HC1, mocetinostat (MGCD0103), droxinostat, MC1568, pracinostat (SB939), divalproex sodium, PCI-34051, givinostat (ITF2357), AR-42, tubastatin A HC1, resminostat, tacedinaline (CI994), RGFP966, HPOB, RG2833 (RGFP109), TMP269, nexturastat A, 4SC-202, LMK-235, or a combination thereof.

5. The composition of claim 2, wherein the histone deacetylase inhibitor is valproic acid, trichostatin A (TSA), M344 (a HDAC inhibitor), sodium phenylbutyrate, entinostat (MS-275), belinostat (PXD101), abexinostat (PCI-24781), dacinostat (LAQ824), quisinostat (JNJ-26481585) 2HC1, mocetinostat (MGCD0103), droxinostat, MC1568, pracinostat (SB939), divalproex sodium, PCI-34051, givinostat (ITF2357), AR-42, tubastatin A HC1, resminostat, tacedinaline (CI994), RGFP966, HPOB, RG2833 (RGFP109), TMP269, nexturastat A, 4SC-202, LMK-235, or a combination thereof.

6. The composition of claim 1, wherein the mitogen-activated extracellular signal-regulated kinase inhibitor is PD0325901, selumetinib (AZD6244), PD184352 (CI-1040), SL-327, refametinib (RDEA119, Bay 86-9766), PD98059, pimasertib (AS-703026), BIX 02188, BIX 02189, PD318088, AZD8330, myricetin, TAK-733, trametinib (GSK1120212), binimetinib (MEK162, ARRY-162, ARRY-438162), GDC-0623, Cobimetinib (GDC-0973, RG7420), APS-2-79 HC1, or a combination thereof.

7. The composition of claim 2, wherein the mitogen-activated extracellular signal-regulated kinase inhibitor is PD0325901, selumetinib (AZD6244), PD184352 (CI-1040), SL-327, refametinib (RDEA119, Bay 86-9766), PD98059, pimasertib (AS-703026), BIX 02188, BIX 02189, PD318088, AZD8330, myricetin, TAK-733, trametinib (GSK1120212), binimetinib (MEK162, ARRY-162, ARRY-438162), GDC-0623, Cobimetinib (GDC-0973, RG7420), APS-2-79 HC1, or a combination thereof.

8. The composition of claim 1, wherein the glycogen synthase kinase-3 inhibitor is CHIR-99021 (CT99021), SB216763, TWS119, indirubin, SB415286, CHIR-98014, Tideglusib, TDZD-8, LY2090314, AZD1080, 1-Azakenpaullone, BIO, AZD2858, AR-A014418, IM-12, bikinin, BIO-Acetoxim, or a combination thereof.

9. The composition of claim 2, wherein the glycogen synthase kinase-3 inhibitor is CHIR-99021 (CT99021), SB216763, TWS119, indirubin, SB415286, CHIR-98014, Tideglusib, TDZD-8, LY2090314, AZD1080, 1-Azakenpaullone, BIO, AZD2858, AR-A014418, IM-12, bikinin, BIO-Acetoxim, or a combination thereof.

10. The composition of claim 1, wherein the transforming growth factor beta receptor 1 (TGF-βR1) inhibitor is RepSox (E-616452), SB-431542, SB-525334, theophylline, SB-505124, galunisertib (LY2157299), GW-788388, pirfenidone, DMH1, LDN-212854, K02288, vactosertib (TEW-7197), SD-208, LDN-214117, SIS3 HC1, LDN-193189 2HC1, or a combination thereof.

11. The composition of claim 2, wherein the transforming growth factor beta receptor 1 (TGF-βR1) inhibitor is RepSox (E-616452), SB-431542, SB-525334, theophylline, SB-505124, galunisertib (LY2157299), GW-788388, pirfenidone, DMH1, LDN-212854, K02288, vactosertib (TEW-7197), SD-208, LDN-214117, SIS3 HC1, LDN-193189 2HC1, or a combination thereof.

12. The composition of claim 1, wherein the inhibitor of histone demethylase is tranylcypromine, GSK J4HC1, IOX1, OG-L002, JIB-04, ML324, GSK-LSD1 2HC1, GSK J1, SP2509, ORY-1001(RG-6016) 2HC1, GSK2879552 2HC1, CPI-455HC1, or a combination thereof.

13. The composition of claim 2, wherein the inhibitor of histone demethylase is tranylcypromine, GSK J4HC1, IOX1, OG-L002, JIB-04, ML324, GSK-LSD1 2HC1, GSK J1, SP2509, ORY-1001(RG-6016) 2HC1, GSK2879552 2HC1, CPI-455HC1, or a combination thereof.

14. The composition of claim 1, wherein the microRNA is miR-291a; and the combination of small-molecule compounds comprises valproic acid, PD0325901, CHIR-99021, E-616452, and tranylcypromine.

15. The composition of claim 2, wherein the microRNA is miR-291a; and the combination of small-molecule compounds comprises valproic acid, PD0325901, CHIR-99021, E-616452, and tranylcypromine.

16. A method for induction of a pluripotent stem cell using the composition of claim 1, the method comprising transfecting the microRNA of the composition into a somatic cell during the reprogramming, and adding synchronously or later the small-molecule compounds of the composition.
